(19) United States
(12) Patent Application Publication
(10) Pub. No.: US 2025/0261929 A1
(43) Pub. Date: Aug. 21, 2025

(54) ULTRASOUND IMAGE CAPTURING APPARATUS, MAMMOGRAPHY APPARATUS, AND CONTROL PROGRAM

(71) Applicant: FUJIFILM Corporation, Tokyo (JP)

(72) Inventors: Hisatsugu HORIUCHI, Kanagawa (JP); Lisako NOBUYAMA, Kanagawa (JP); Hiroki NAKAYAMA, Kanagawa (JP); Takeyasu KOBAYASHI, Kanagawa (JP); Sayaka SAITO, Kanagawa (JP)

(73) Assignee: FUJIFILM Corporation, Tokyo (JP)

(21) Appl. No.: 19/038,805

(22) Filed: Jan. 28, 2025

(30) Foreign Application Priority Data
Feb. 15, 2024 (JP) 2024-021456

(51) Int. Cl. A61B 8/00; A61B 8/08

(52) U.S. Cl. CPC A61B 8/406; A61B 8/0825; A61B 8/403

(57) ABSTRACT

An ultrasound image capturing apparatus includes an imaging table in which a transducer that transmits an ultrasonic wave toward an imaging surface, to capture an ultrasound image of a breast, and a transducer movement unit that moves the transducer in a direction along the imaging surface are built in advance, in which the imaging surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets.

### CROSS-REFERENCE TO RELATED APPLICATIONS

This application claims priority under 35 USC 119 from Japanese Patent Application No. 2024-021456 filed on Feb. 15, 2024, the disclosure of which is incorporated by reference herein.

### BACKGROUND

### 1. Technical Field

The present disclosure relates to an ultrasound image capturing apparatus, a mammography apparatus, and a control program.

### 2. Related Art

An ultrasound image capturing apparatus that captures an ultrasound image of a breast of a subject by scanning a transducer that outputs ultrasonic waves along the breast is known.

JP2008-173291A discloses a medical imaging apparatus that captures an ultrasound image of a breast placed on a transducer array disposed on an imaging table from a contact surface in which the breast is in contact with the transducer array and that captures a radiation image of the breast, by using the transducer array.

### SUMMARY

In a case in which the radiation image of the breast compressed by a compression plate is captured by the medical imaging apparatus such as JP2008-173291A, an image of the transducer array is reflected in the radiation image in a case in which the transducer array is disposed on the imaging table. Therefore, the medical imaging apparatus may include a movement mechanism that retracts the transducer array from above the imaging table.

However, in a case in which the transducer array is retracted by the movement mechanism, the transducer array in close contact with the breast is not present, and thus a compression state of the breast is changed.

In order to suppress the change in the compression state of the breast accompanying the movement of the transducer array, the transducer array need only be built inside the imaging table instead of being disposed on the imaging table, but it is necessary to adjust an acoustic impedance among the transducer, the imaging table, and the breast via the transducer built in the imaging table. Further, since the breast is pressed against the imaging table by the compression plate, the contact surface with the breast on the imaging table is required to have rigidity.

The present disclosure has been made in consideration of the above-described circumstances, and an object of the present disclosure is to provide an ultrasound image capturing apparatus, a mammography apparatus, and a control program capable of achieving acoustic matching with a breast while ensuring the rigidity of a contact surface of an imaging table in contact with the breast.

A first aspect of the technology of the present disclosure relates to an ultrasound image capturing apparatus comprising: an imaging table in which a first transducer that transmits an ultrasonic wave toward a contact surface in contact with a breast, to capture an ultrasound image of the breast, and a movement mechanism that moves the first transducer in a direction along the contact surface with the breast are built in advance, in which the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets.

A second aspect of the technology of the present disclosure relates to the ultrasound image capturing apparatus according to the first aspect, in which the first transducer has a shape that extends longer in a chest wall surface direction along a chest wall of a subject than in a front-rear direction along a direction intersecting the chest wall of the subject, and the movement mechanism moves the first transducer in the front-rear direction.

A third aspect of the technology of the present disclosure relates to the ultrasound image capturing apparatus according to the second aspect, in which the movement mechanism further moves the first transducer in the chest wall surface direction.

A fourth aspect of the technology of the present disclosure relates to the ultrasound image capturing apparatus according to the first aspect, in which the first transducer has a shape that extends longer in a front-rear direction along a direction intersecting a chest wall of a subject than in a chest wall surface direction along the chest wall of the subject, and the movement mechanism moves the first transducer in the chest wall surface direction.

A fifth aspect of the technology of the present disclosure relates to the ultrasound image capturing apparatus according to the fourth aspect, in which the movement mechanism further moves the first transducer in the front-rear direction.

A sixth aspect of the technology of the present disclosure relates to the ultrasound image capturing apparatus according to the first aspect, in which, in the contact surface of the imaging table, the carbon fiber sheets are laminated such that the acoustic impedance is decreased from a carbon fiber sheet in contact with the first transducer toward a carbon fiber sheet in contact with the breast.

A seventh aspect of the technology of the present disclosure relates to the ultrasound image capturing apparatus according to the sixth aspect, in which a fiber direction of at least one carbon fiber sheet among the plurality of laminated carbon fiber sheets is disposed along a chest wall surface direction along a chest wall of a subject.

An eighth aspect of the technology of the present disclosure relates to the ultrasound image capturing apparatus according to the seventh aspect, in which the fiber direction of each of the carbon fiber sheet in contact with the first transducer and the carbon fiber sheet in contact with the breast is disposed along the chest wall surface direction.

A ninth aspect of the technology of the present disclosure relates to the ultrasound image capturing apparatus according to the first aspect, further comprising: a second transducer that captures an ultrasound image of the breast from a compression surface of a compression plate that compresses the breast to bring the breast into close contact with the contact surface of the imaging table; and a controller that captures the ultrasound images of the breast by using the first transducer and the second transducer.

A tenth aspect of the technology of the present disclosure relates to the ultrasound image capturing apparatus according to the ninth aspect, in which the controller switches an imaging mode of the ultrasound image of the breast by controlling imaging ranges of the first transducer and the second transducer.

An eleventh aspect of the technology of the present disclosure relates to the ultrasound image capturing apparatus according to the tenth aspect, in which the controller sets a boundary surface between the contact surface of the imaging table and the compression surface of the compression plate at a predetermined distance from the contact surface of the imaging table, and then performs control of the imaging ranges such that the first transducer and the second transducer image the same range of the breast and performs control of combining the ultrasound image, which is captured by the first transducer, from the contact surface of the imaging table to the boundary surface and the ultrasound image, which is captured by the second transducer, from the compression surface of the compression plate to the boundary surface, to generate one ultrasound image of the same breast.

A twelfth aspect of the technology of the present disclosure relates to the ultrasound image capturing apparatus according to the tenth aspect, in which the controller performs control of the imaging ranges such that the first transducer and the second transducer image different ranges.

A thirteenth aspect of the technology of the present disclosure relates to a mammography apparatus comprising: the ultrasound image capturing apparatus according to any one of the first to twelfth aspects.

A fourteenth aspect of the technology of the present disclosure relates to the mammography apparatus according to the thirteenth aspect, in which a grid that is provided between the contact surface of the imaging table and a radiation detector and that reduces an amount of scattered rays, which are generated by scattering of radiation emitted from a radiation source via the breast, incident on the radiation detector as compared to before installation is used as the movement mechanism.

A fifteenth aspect of the technology of the present disclosure relates to the mammography apparatus according to the fourteenth aspect, in which the imaging table includes an attachment/detachment device that attaches the first transducer to the grid in a case in which the grid is moved to a predetermined position and that detaches the first transducer from the grid in a case in which the grid to which the first transducer is attached returns to the position.

A sixteenth aspect of the technology of the present disclosure relates to a control program for causing a computer to execute a process, with respect to an ultrasound image capturing apparatus including an imaging table in which a first transducer that transmits an ultrasonic wave toward a contact surface in contact with a breast, to capture an ultrasound image of the breast, and a movement mechanism that moves the first transducer are built in advance, in which the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets, the process comprising: controlling the movement mechanism to move the first transducer in a direction along the contact surface of the imaging table with the breast and controlling the first transducer to capture the ultrasound image of the breast from the contact surface of the imaging table with the breast.

According to the present disclosure, it is possible to achieve the acoustic matching with the breast while ensuring the rigidity of the contact surface of the imaging table in contact with the breast.

### BRIEF DESCRIPTION OF THE DRAWINGS

Exemplary embodiments of the technology of the disclosure will be described in detail based on the following figures, wherein:

FIG. 1 is a diagram showing a configuration example of a medical imaging system according to a first embodiment;

FIG. 2 is a diagram showing a functional configuration example of an ultrasound image capturing apparatus;

FIG. 3 is a diagram showing a functional configuration example of an image storage system;

FIG. 4 is a diagram showing an appearance example of the ultrasound image capturing apparatus according to the first embodiment as seen from a side surface;

FIG. 5 is a diagram showing an example in which an imaging surface is seen from a position above the imaging surface facing the imaging surface;

FIG. 6 is a diagram showing another example of attachment of a transducer to a movement table;

FIG. 7 is a diagram showing a cross-sectional example of the imaging surface;

FIG. 8 is a diagram showing an example of a fiber direction of carbon fiber sheets;

FIG. 9 is a flowchart showing an example of a flow of imaging processing executed by the ultrasound image capturing apparatus;

FIG. 10 is a diagram showing an appearance example in a case in which an ultrasound image capturing apparatus according to a modification example of the first embodiment is seen from a side surface;

FIG. 11 is a diagram showing a control example of an imaging range of the transducer;

FIG. 12 is a flowchart showing another example of the flow of the imaging processing executed by the ultrasound image capturing apparatus;

FIG. 13 is a diagram showing an example of an ultrasound image captured in a high definition mode;

FIG. 14 is a diagram showing another control example of the imaging range of the transducer;

FIG. 15 is a diagram showing an example of an ultrasound image captured in a high speed mode;

FIG. 16 is a diagram showing a configuration example of a medical imaging system according to a second embodiment;

FIG. 17 is a diagram showing a functional configuration example of a mammography apparatus and a console;

FIG. 18 is a diagram showing an appearance example in a case in which the mammography apparatus is seen from a side surface;

FIG. 19 is a diagram showing an operation example of an attachment/detachment device; and

FIG. 20 is a flowchart showing an example of a flow of imaging processing executed by the mammography apparatus.

### DETAILED DESCRIPTION

Hereinafter, the present embodiment of the present invention will be described in detail with reference to the accompanying drawings. It should be noted that the same components and the same processing are denoted by the same reference numerals throughout all of the drawings, and redundant description will be omitted. Dimensional ratios in the drawings are exaggerated for convenience of description, and may be different from the actual ratios.

### First Embodiment

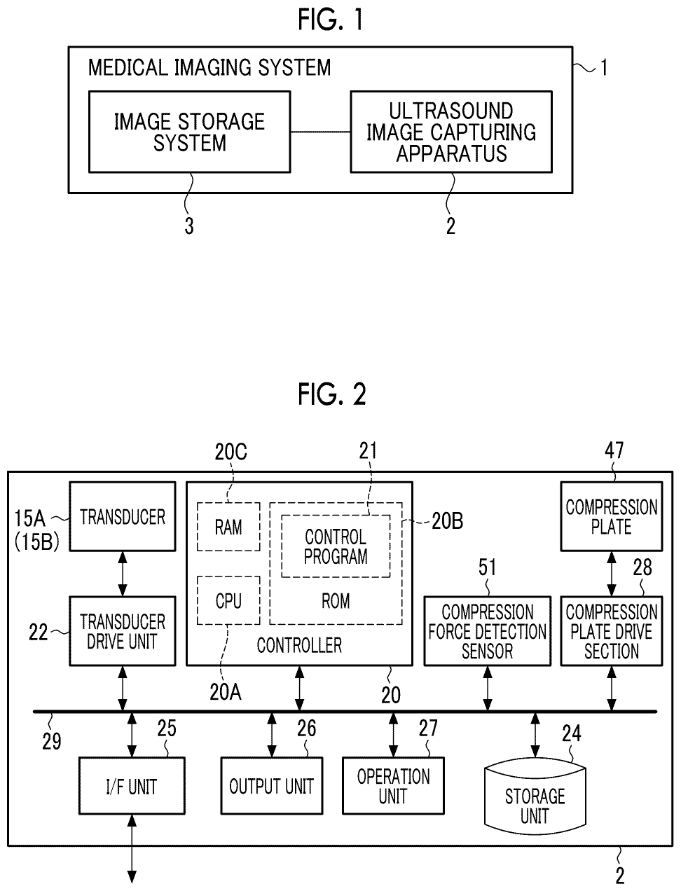

FIG. 1 is a diagram showing a configuration example of a medical imaging system 1 according to the first embodiment. The medical imaging system 1 includes an ultrasound image capturing apparatus 2 and an image storage system 3.

The ultrasound image capturing apparatus 2 is an apparatus that captures an ultrasound image of a breast of a subject as an examination subject by, for example, a medical staff member such as an examination technician or a doctor.

The image storage system 3 is a system that stores the ultrasound images captured by the ultrasound image capturing apparatus 2. The image storage system 3 extracts an ultrasound image corresponding to a request from the ultrasound image capturing apparatus 2 or a console 6 (see FIG. 16) described later from among the stored ultrasound images, and transmits the extracted ultrasound image to a request source apparatus. Specific examples of the image storage system 3 include a picture archiving and communication system (PACS).

First, a functional configuration example of the ultrasound image capturing apparatus 2 will be described. FIG. 2 is a diagram showing a functional configuration example of the ultrasound image capturing apparatus 2.

As shown in FIG. 2, the ultrasound image capturing apparatus 2 includes a controller 20, a transducer drive unit 22, a transducer 15A, a storage unit 24, an interface (I/F) unit 25, an output unit 26, an operation unit 27, a compression plate drive section 28, a compression plate 47, and a compression force detection sensor 51. The controller 20, the transducer drive unit 22, the storage unit 24, the I/F unit 25, the output unit 26, the operation unit 27, the compression plate drive section 28, and the compression force detection sensor 51 are connected to each other via a bus 29 such that various types of information can be exchanged.

The controller 20 controls the operation of the ultrasound image capturing apparatus 2 based on an instruction from the medical staff member. The controller 20 comprises a central processing unit (CPU) 20A as an example of a processor, a read-only memory (ROM) 20B, and a random-access memory (RAM) 20C. The ROM 20B stores in advance various programs including a control program 21 that is read by the CPU 20A to perform control for the capturing of an ultrasound image, and various parameters to be referred to in a case in which the CPU 20A controls the operation of the ultrasound image capturing apparatus 2. The RAM 20C is used as a transitory work area of the CPU 20A.

The transducer 15A outputs ultrasonic waves to the examination subject, that is, the breast, acquires reflected waves of the ultrasonic waves reflected by the breast, converts a state of the reflected waves into reflected wave data from the acquired reflected waves, and outputs the reflected wave data to the controller 20. The controller 20 that receives the reflected wave data generates the ultrasound image of the breast by using the received reflected wave data. The transducer 15A is an example of a first transducer according to the present disclosure.

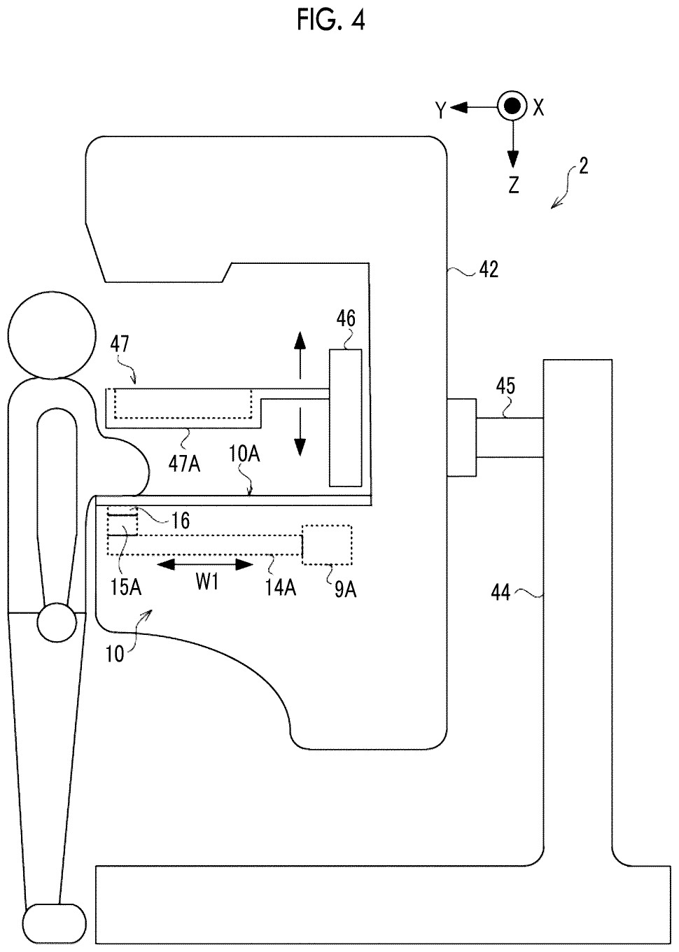

The transducer drive unit 22 controls a transducer movement unit 9A (see FIG. 4) described later in response to an instruction from the controller 20, and moves the transducer 15A in at least one of a chest wall surface direction, that is, a direction along a chest wall formed by the chest of the subject, or a front-rear direction, that is, a direction intersecting the chest wall of the subject.

The compression plate 47 is an instrument that presses a compression surface 47A against the breast to compress the breast. It is preferable that the compression plate 47 has transparency such that a compression state of the breast can be visually recognized.

The compression plate drive section 28 moves the compression plate 47 in a compression direction and a compression release direction under the control of the controller 20. The compression direction is a direction in which the breast of the subject is pressed against an imaging table 10 (see FIG. 4) described later, and the compression release direction is a direction in which the compression of the breast via the compression plate 47 is released.

The compression force detection sensor 51 has a function of detecting a compression force of the compression plate 47, which is moved by driving of the compression plate drive section 28, against the breast.

As described above, the ultrasound image capturing apparatus 2 captures the ultrasound image of the breast in a state of being compressed by the compression plate 47. It should be noted that the ultrasound image of the breast can be captured without compressing the breast, and thus the ultrasound image capturing apparatus 2 need not comprise the compression plate drive section 28, the compression plate 47, and the compression force detection sensor 51.

The storage unit 24 stores the captured ultrasound images, various types of other information, and the like. The storage unit 24 is an example of a storage device that holds the stored information even in a case in which the power supplied to the storage unit 24 is cut off, and, for example, a semiconductor memory, such as a solid state drive (SSD), is used, or a hard disk may be used.

The I/F unit 25 transmits and receives various types of information to and from an external apparatus connected to a communication line (not shown) such as a local area network (LAN), via wireless communication or wired communication. For example, the controller 20 transmits the captured ultrasound image to the image storage system 3 via the I/F unit 25.

The output unit 26 outputs, to the medical staff member, information processed by the controller 20 such as information related to an imaging status of the ultrasound image, the captured ultrasound image, and a warning. Outputting the information means making the information recognizable by the medical staff member. Therefore, for example, a form in which information is displayed on a display (not shown) as an example of a display device, a form in which information is printed on a recording medium such as paper by an image forming apparatus (not shown), and a form in which notification of information is issued by voice via a speaker (not shown) are all examples of the information output by the output unit 26. It should be noted that a form in which the information is transmitted to the external apparatus via the I/F unit 25 is also the output example of the information.

The operation unit 27 is used by the medical staff member to input, for example, instructions and various types of information related to the capturing of the ultrasound image. There is no restriction on an operation form in the operation unit 27, and, for example, the operation can be received by a switch, a touch panel, an electronic pen, a keyboard, a voice, a mouse, or the like.

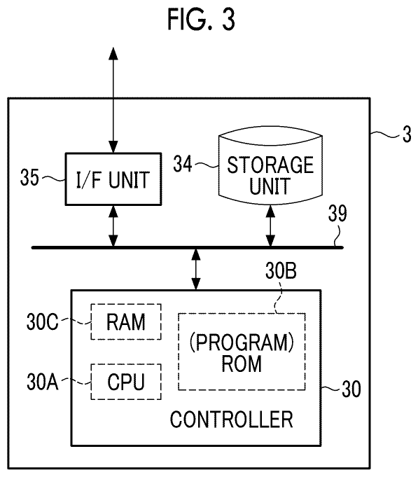

Meanwhile, FIG. 3 is a diagram showing a functional configuration example of the image storage system 3. As shown in FIG. 3, the image storage system 3 includes a controller 30, a storage unit 34, and an I/F unit 35. The controller 30, the storage unit 34, and the I/F unit 35 are connected to each other via a bus 39 such that various types of information can be exchanged.

The controller 30 controls the operation of the image storage system 3. The controller 30 comprises a CPU 30A, a ROM 30B, and a RAM 30C. The ROM 30B stores in advance various programs that are read by the CPU 30A to perform control for storage of the ultrasound image, and various parameters to be referred to in a case in which the CPU 30A controls the operation of the image storage system 3. The RAM 30C is used as a transitory work area of the CPU 30A.

The storage unit 34 stores the ultrasound image in association with, for example, an imaging order or information related to the subject. That is, the storage unit 34 functions as a database for the ultrasound image.

The I/F unit 35 transmits and receives various types of information to and from the external apparatus connected to a communication line such as the LAN, via wireless communication or wired communication. For example, the controller 30 transmits the requested ultrasound image to the ultrasound image capturing apparatus 2 via the I/F unit 35.

Hereinafter, an example of the capturing of the ultrasound image of the breast via the ultrasound image capturing apparatus 2 will be described. FIG. 4 is a diagram showing an appearance example of the ultrasound image capturing apparatus 2 according to the first embodiment as seen from a side surface.

The ultrasound image capturing apparatus 2 comprises an arm part 42, a base 44, and a shaft part 45. The arm part 42 is held by the base 44 to be movable in an up-down direction (Z axis direction). The shaft part 45 connects the arm part 42 to the base 44. The arm part 42 can be rotated relative to the base 44 with the shaft part 45 as a rotation axis. A compression plate drive unit 46 is provided in the arm part 42.

The imaging table 10 is provided below the arm part 42, and the subject places the breast on an imaging surface 10A provided on an upper surface of the imaging table 10. The imaging surface 10A of the imaging table 10 in contact with the breast is an example of a contact surface with the breast of the imaging table 10 according to the present disclosure.

The compression plate 47 is attached to the compression plate drive unit 46, and the compression plate drive unit 46 moves the compression plate 47 in the compression direction (downward in the example of FIG. 4) in response to an instruction from the compression plate drive section 28, to press and compress the breast placed on the imaging surface 10A of the imaging table 10 against the imaging surface 10A. In addition, the compression plate drive unit 46 moves the compression plate 47, which compresses the breast, in the compression release direction (upward in the example of FIG. 4) in response to the instruction from the compression plate drive section 28, to release the compression of the breast via the compression plate 47.

Meanwhile, the transducer 15A that transmits the ultrasonic waves toward the imaging surface 10A and captures the ultrasound image of the breast pressed against the imaging surface 10A by the compression plate 47 is built in the imaging table 10. A transducer cover 16 is attached to a surface of the transducer 15A facing the imaging surface 10A. The transducer cover 16 functions as a lubricating member that reduces a contact resistance between a back side of the imaging surface 10A and the transducer 15A, and functions as an acoustic matching member that reduces a difference in acoustic impedance between different objects, such as the imaging surface 10A and the transducer 15A.

The transducer 15A is attached to a movement table 14A that is also built in the imaging table 10 in advance, and the transducer movement unit 9A moves the movement table 14A in the front-rear direction (in the example of FIG. 4, a direction represented by an arrow W1) under the control of the controller 20 via the transducer drive unit 22. Therefore, the transducer 15A attached to the movement table 14A is also moved in the front-rear direction in accordance with the movement of the movement table 14A. The movement table 14A and the transducer movement unit 9A according to the present disclosure are examples of a movement mechanism that moves the transducer 15A inside the imaging table 10.

It should be noted that a target to be moved by the transducer movement unit 9A is not limited to a plate-like object such as the movement table 14A. Any shape may be used as long as the transducer 15A can be moved and it is an object to which the transducer 15A is attached, and other shapes, such as a rod shape and a lattice shape, may be used.

Since the transmission position of the ultrasonic waves is also moved by moving the transducer 15A in the front-rear direction, the ultrasound image of the entire breast placed on the imaging surface 10A is obtained. It should be noted that moving the transducer 15A in the direction along the imaging surface 10A via the movement mechanism may be referred to as “scanning”.

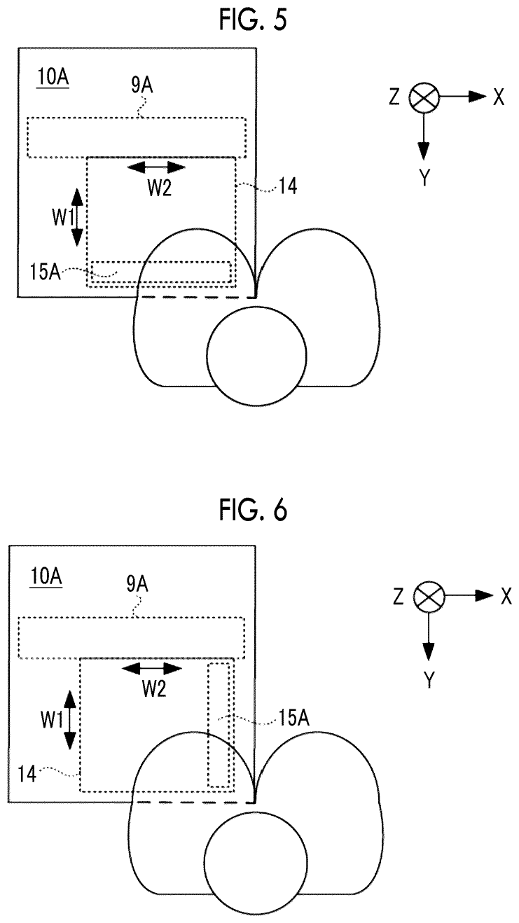

FIG. 5 is a diagram showing an example in which the subject who places the breast on the imaging surface 10A is seen from a position above the imaging surface 10A facing the imaging surface 10A of the imaging table 10. For convenience of description, in FIG. 5, the transducer cover 16 and the compression plate 47 are not shown.

As shown in FIG. 5, the transducer 15A has a rectangular parallelepiped shape and is attached to the movement table 14A such that a longitudinal direction of the transducer 15A is along the chest wall surface direction represented by an arrow W2. That is, the transducer 15A is attached to the movement table 14A such that a length in the chest wall surface direction is longer than a length in the front-rear direction represented by the arrow W1.

It is preferable that the transducer 15A is attached to an end part of the movement table 14A that is closest to the subject so that the transducer 15A can scan the entire range in which the breast is placed on the imaging surface 10A.

It should be noted that the transducer movement unit 9A may further move the transducer 15A in the chest wall surface direction while moving the transducer 15A in the front-rear direction.

In addition, the attachment direction of the transducer 15A to the subject is not limited to the form of the attachment in a direction in which the longitudinal direction of the transducer 15A as shown in FIG. 5 is along the chest wall surface direction.

FIG. 6 is a diagram showing another example of the attachment of the transducer 15A to the movement table 14A. For convenience of description, in FIG. 6, the transducer cover 16 and the compression plate 47 are not shown.

As shown in FIG. 6, the transducer 15A may be attached to the movement table 14A such that the longitudinal direction of the transducer 15A is along the front-rear direction represented by the arrow W1. That is, the transducer 15A is attached to the movement table 14A such that the length in the front-rear direction is longer than the length in the chest wall surface direction represented by the arrow W2. Thereafter, the transducer movement unit 9A moves the movement table 14A in the chest wall surface direction. In accordance with this, since the transducer 15A attached to the movement table 14A is also moved in the chest wall surface direction in accordance with the movement of the movement table 14A, the ultrasound image of the entire breast placed on the imaging surface 10A is obtained.

It should be noted that the transducer movement unit 9A may further move the transducer 15A in the front-rear direction while moving the transducer 15A in the chest wall surface direction.

In the following description, as shown in FIG. 5, the transducer 15A is assumed to be attached to the movement table 14A such that the longitudinal direction of the transducer 15A is along the chest wall surface direction.

Hereinafter, a structure of the imaging surface 10A in the imaging table 10 will be described. In the capturing of the ultrasound image, the breast is pressed against the imaging surface 10A by the compression plate 47, and thus the imaging surface 10A is required to have rigidity such that the imaging surface 10A is not bent even in a case in which the breast is pressed against the imaging surface 10A. Therefore, a carbon fiber sheet that has a specific gravity of about ⅕ of iron and a tensile strength of about 10 times that of steel and that is used as a reinforcing material for various structures is used for the imaging surface 10A.

However, since the carbon fiber sheets have the same type, the carbon fiber sheets have a unique acoustic impedance, and thus, in a case in which a single layer of the carbon fiber sheet is used, it is only possible to select a carbon fiber sheet having an acoustic impedance close to the acoustic impedance of any one of the breast or the transducer 15A having different acoustic impedances. That is, it is difficult to form the imaging surface 10A, which has balance between both the rigidity and the acoustic matching performance, with a single layer of the carbon fiber sheet.

Therefore, the imaging surface 10A of the imaging table 10 according to the present disclosure is composed of a plurality of laminated carbon fiber sheets having different acoustic impedances.

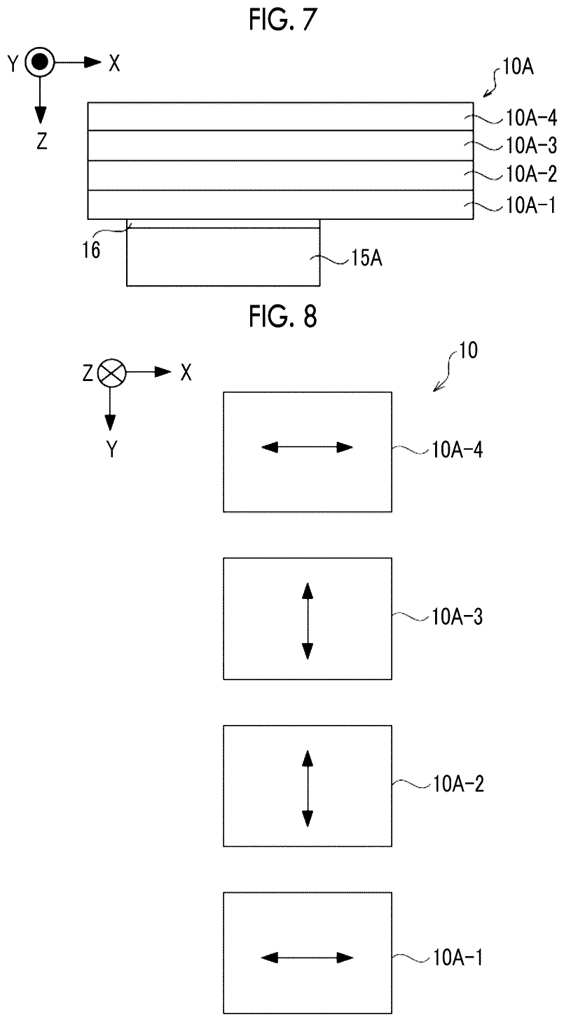

FIG. 7 is a diagram showing a cross-sectional example of the imaging surface 10A. The imaging surface 10A shown in FIG. 7 is composed of four carbon fiber sheets 10A-1 to 10A-4 laminated in the up-down direction (Z axis direction in the example of FIG. 7). Among the carbon fiber sheets 10A-1 to 10A-4 constituting the imaging surface 10A, the carbon fiber sheet 10A-1 is in contact with the transducer 15A (specifically, is in contact with the transducer cover 16 of the transducer 15A), and the carbon fiber sheet 10A-4 is in contact with the breast.

The acoustic impedances of the four carbon fiber sheets 10A-1 to 10A-4 are different from each other, and the carbon fiber sheets 10A-1 to 10A-4 are laminated such that the acoustic impedance is decreased from the carbon fiber sheet 10A-1 to the carbon fiber sheet 10A-4. That is, the carbon fiber sheets 10A-1 to 10A-4 are laminated such that the acoustic impedance of the carbon fiber sheet 10A-2 is lower than the acoustic impedance of the carbon fiber sheet 10A-1, the acoustic impedance of the carbon fiber sheet 10A-3 is lower than the acoustic impedance of the carbon fiber sheet 10A-2, and the acoustic impedance of the carbon fiber sheet 10A-4 is lower than the acoustic impedance of the carbon fiber sheet 10A-3.

The reason for laminating the carbon fiber sheets 10A-1 to 10A-4 in this way is that the acoustic impedance of the human body is lower than the acoustic impedance of the transducer 15A. For example, the acoustic impedance of the transducer 15A is in a range of 20 [MRayls] or more and 38 [MRayls] or less, whereas the acoustic impedance of the human body is in a vicinity of 1.5 [MRayls]. Since 1 [Rays] is 1 [kg/m² s], 1 [MRays] is 1×10⁶ [kg/m² s]. It should be noted that the term “vicinity” refers to a range that can be regarded as the displayed value, and represents, for example, a range having a width of ±α% with respect to the displayed value. The value of a is set depending on the situation.

As the acoustic impedances between different objects approach each other, an amount of reflection of the ultrasonic waves generated at a boundary between the objects is decreased, and thus a clear ultrasound image is obtained. Therefore, it is preferable that the acoustic impedance of the carbon fiber sheet 10A-1 in contact with the transducer 15A is made close to the acoustic impedance of the transducer 15A, and the acoustic impedance of the carbon fiber sheet 10A-4 in contact with the breast is made close to the acoustic impedance of the human body, so that the carbon fiber sheets 10A-1 to 10A-4 are laminated such that the acoustic impedance is decreased from the carbon fiber sheet 10A-1 toward the carbon fiber sheet 10A-4. That is, the acoustic impedance is made to approach the acoustic impedance of the human body, from the transducer 15A toward the human body.

Specifically, it is preferable that the acoustic impedance of the carbon fiber sheet 10A-1 is in a vicinity of 20 [MRayls], the acoustic impedance of the carbon fiber sheet 10A-2 is 5 [MRayls] or more and 15 [MRayls] or less, the acoustic impedance of the carbon fiber sheet 10A-3 is 1.5 [MRayls] or more and 5 [MRayls] or less, and the acoustic impedance of the carbon fiber sheet 10A-4 is in the vicinity of 1.5 [MRayls]. The acoustic impedance can be measured by using, for example, an acoustic impedance measurement device.

It should be noted that, in the imaging surface 10A, it is preferable that at least the acoustic impedance of the carbon fiber sheet 10A-1 in contact with the transducer 15A is included in the range of the acoustic impedance that can be taken by the transducer 15A. This is because the acoustic impedance of the transducer 15A is not changed in the middle unless the type of the transducer 15A to be used is changed, but the acoustic impedance of the breast varies depending on the person. Therefore, the acoustic matching between the transducer 15A and the carbon fiber sheet 10A-1 in contact with the transducer 15A is more easily achieved than the acoustic matching between the transducer 15A and the carbon fiber sheet 10A-4.

In a case in which the carbon fiber sheets 10A-1 to 10A-4 are sheets formed by braiding carbon fibers in one direction, the sheets are more likely to be deflected in a case in which a load is applied along the fiber direction than in a case in which a load is applied along a direction intersecting the fiber direction.

For example, in a case in which a situation is considered in which the breast is pressed against the carbon fiber sheet 10A-4 by the compression plate 47, the breast is pressed against the end part of the imaging surface 10A in the front-rear direction and is pressed against a central portion of the imaging surface 10A in the chest wall surface direction, so that the load is likely to be applied in the chest wall surface direction rather than the front-rear direction.

Therefore, by disposing the carbon fiber sheet 10A-N (N is an integer of 1 to 4) among the carbon fiber sheets 10A-1 to 10A-4 such that the fiber direction in at least one carbon fiber sheet 10A-N is along the chest wall surface direction of the subject, the deflection in the chest wall surface direction can be reduced as compared to a case in which the carbon fiber sheet 10A-N is disposed such that the fiber direction is along the front-rear direction of the subject, and the rigidity of the imaging surface 10A can be ensured.

FIG. 8 is a view showing an example of the fiber direction of each of the carbon fiber sheets 10A-1 to 10A-4. FIG. 8 shows a state in which the carbon fiber sheets 10A-1 to 10A-4 constituting the imaging surface 10A are virtually shifted horizontally without changing a disposition direction, and the carbon fiber sheets 10A-1 to 10A-4 are seen from an upper position facing each sheet surface. The directions of the arrows in the carbon fiber sheets 10A-1 to 10A-4 indicate the fiber directions of the carbon fibers in each of the sheets. It should be noted that, in the example of FIG. 8, an X axis direction represents the chest wall surface direction, and a Y axis direction represents the front-rear direction. That is, in the example of FIG. 8, the subject stands toward a long side of the carbon fiber sheets 10A-1 to 10A-4, and the breast is placed on the imaging surface 10A from the long side of the carbon fiber sheets 10A-1 to 10A-4.

In the examples of the carbon fiber sheets 10A-1 to 10A-4 shown in FIG. 8, the fiber directions of the carbon fiber sheet 10A-1 and the carbon fiber sheet 10A-4 constituting the outermost layer of the imaging surface 10A are disposed along the chest wall surface direction, and the fiber directions of the carbon fiber sheet 10A-2 and the carbon fiber sheet 10A-3 constituting the inner layer of the imaging surface 10A are disposed along the front-rear direction.

It should be noted that the fiber direction of the carbon fibers in the carbon fiber sheets 10A-1 to 10A-4 is not limited to the example of FIG. 8. Among the plurality of laminated carbon fiber sheets 10A-1 to 10A-4, the fiber direction of at least one carbon fiber sheet 10A-N need only be disposed along the chest wall surface direction. Therefore, for example, in the example shown in FIG. 8, the fiber direction of any one of the carbon fiber sheet 10A-1 or the carbon fiber sheet 10A-4 need only be disposed along the chest wall surface direction.

Further, the carbon fiber sheet 10A-2 and the carbon fiber sheet 10A-3 constituting the inner layer of the imaging surface 10A may be disposed such that the fiber direction is along the chest wall surface direction, and the carbon fiber sheet 10A-1 and the carbon fiber sheet 10A-4 constituting the outermost layer of the imaging surface 10A may be disposed such that the fiber direction is along the front-rear direction.

As described above, by disposing the fiber direction of at least one carbon fiber sheet 10A-N among the plurality of laminated carbon fiber sheets 10A-1 to 10A-4 along the chest wall surface direction, the deflection of the imaging surface 10A in the chest wall surface direction can be reduced as compared to a case in which the fiber directions of all the carbon fiber sheets 10A-1 to 10A-4 are disposed along the front-rear direction, so that the rigidity of the imaging surface 10A can be ensured.

It should be noted that, for example, all the carbon fiber sheets 10A-1 to 10A-4 may be disposed such that the fiber directions are along the chest wall surface direction, but, as shown in FIG. 8, in a case in which the imaging surface 10A is composed such that at least one carbon fiber sheet 10A-N of which the fiber direction is along the chest wall surface direction and at least one carbon fiber sheet 10A-N of which the fiber direction is along the front-rear direction are disposed, both the deflection of the imaging surface 10A along the chest wall surface direction and the deflection of the imaging surface 10A along the front-rear direction can be reduced, so that the rigidity of the imaging surface 10A can be further ensured. Therefore, the imaging surface 10A may be composed of the carbon fiber sheets 10A-1 to 10A-4 in which the fiber directions are alternately changed such that the fiber directions of the adjacent carbon fiber sheets 10A-N are different from each other.

In addition, since the outermost layer of the imaging surface 10A, which is in contact with the breast or the transducer 15A, is more likely to be loaded in the chest wall surface direction than the inner layer of the imaging surface 10A, as shown in FIG. 8, it is preferable that the carbon fiber sheet 10A-1 and the carbon fiber sheet 10A-4 constituting the outermost layer of the imaging surface 10A are disposed such that the fiber directions are along the chest wall surface direction.

It goes without saying that the number of layers of the carbon fiber sheet 10A-N constituting the imaging surface 10A is not limited to four layers. In a case in which the number of layers of the carbon fiber sheet 10A-N constituting the imaging surface 10A is two or more, the imaging surface 10A can achieve the acoustic matching with the breast while ensuring the rigidity that can withstand the load generated by the compression of the breast by the compression plate 47.

Hereinafter, the operation of the ultrasound image capturing apparatus 2 shown in FIG. 4 that captures the ultrasound image of the breast placed on the imaging surface 10A of the imaging table 10 using the transducer 15A will be described.

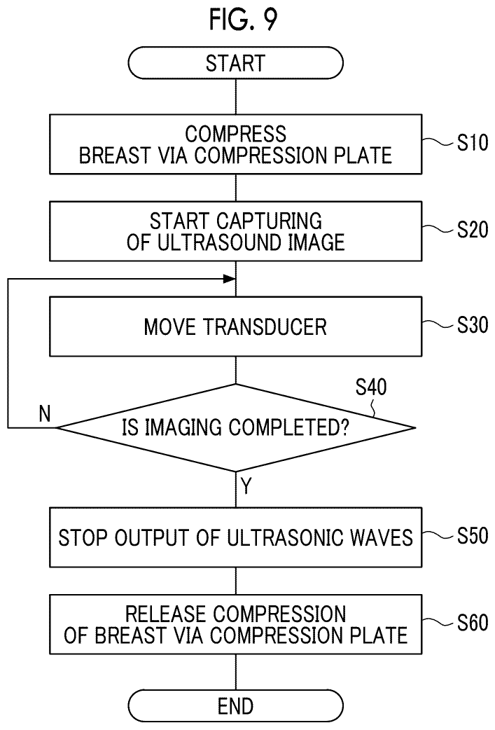

FIG. 9 is a flowchart showing an example of a flow of imaging processing executed by the ultrasound image capturing apparatus 2 in a case in which an instruction to start the capturing of the ultrasound image of the breast is received through an operation via the operation unit 27 performed by the medical staff member. The CPU 20A of the ultrasound image capturing apparatus 2 reads the control program 21 from the ROM 20B and executes the imaging processing. It is assumed that the subject has already placed the breast, which is the examination subject, on the imaging surface 10A of the imaging table 10.

First, in step S10, the controller 20 performs control of the compression plate drive unit 46 via the compression plate drive section 28, and moves the compression plate 47 in the compression direction to compress the breast with the compression plate 47 and press the breast against the imaging surface 10A.

In step S20, the controller 20 performs control of the transducer 15A to output the ultrasonic waves from the transducer 15A and starts the capturing of the ultrasound image of the breast.

In step S30, the controller 20 moves the transducer 15A by controlling the transducer movement unit 9A via the transducer drive unit 22. Therefore, the controller 20 can capture continuous ultrasound images of the entire breast.

In step S40, the controller 20 determines whether the capturing of the ultrasound images in a planned range is completed. In a case in which the imaging is not yet completed, the processing proceeds to step S30, and the processing of moving the transducer 15A to the designated position is repeatedly executed.

On the other hand, in a case in which it is determined by the determination processing of step S40 that the capturing of ultrasound images is completed, the processing proceeds to step S50.

In step S50, the controller 20 performs control of the transducer 15A to stop the output of the ultrasonic waves.

In step S60, the controller 20 moves the compression plate 47 in the compression release direction and releases the compression of the breast via the compression plate 47 by controlling the compression plate drive unit 46 via the compression plate drive section 28. In this way, the imaging processing shown in FIG. 9 ends.

As described above, with the ultrasound image capturing apparatus 2 according to the first embodiment, since the transducer 15A that is movable in at least one of the front-rear direction or the chest wall surface direction is built in the imaging table 10, it is possible to capture the ultrasound image of the breast in contact with the imaging surface 10A from the imaging surface 10A without placing an auxiliary table such as an enlarged imaging table (not shown) on the imaging surface 10A.

In addition, since the imaging surface 10A is composed of the plurality of laminated carbon fiber sheets 10A-N having different acoustic impedances, and the fiber direction in at least one carbon fiber sheet 10A-N among the plurality of carbon fiber sheets 10A-Nis disposed along the chest wall surface direction, the imaging surface 10A can achieve the acoustic matching with the breast while ensuring the rigidity capable of withstanding the load generated by the compression of the breast by the compression plate 47, as compared to a case in which the imaging surface 10A is composed of a single layer.

### Modification Example of First Embodiment

In the first embodiment, the ultrasound image of the breast in contact with the imaging surface 10A from the imaging surface 10A side is captured by using the transducer 15A built in the imaging table 10. In the present modification example, an ultrasound image capturing apparatus 2A that captures the ultrasound image of the breast from a plurality of surfaces will be described.

It should be noted that a functional configuration example of the ultrasound image capturing apparatus 2A is the same as the functional configuration example of the ultrasound image capturing apparatus 2 shown in FIG. 2.

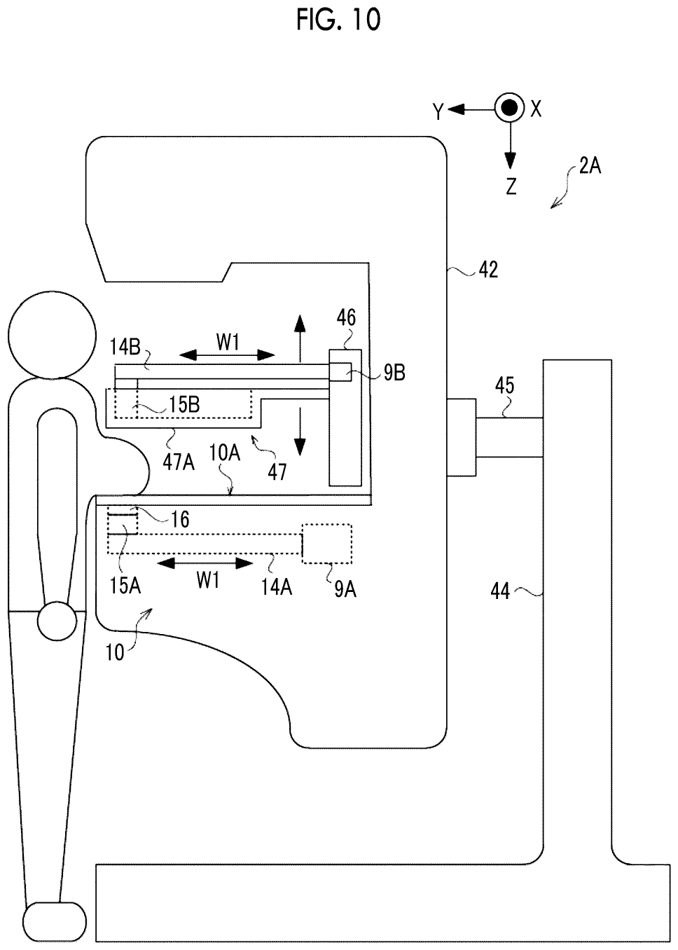

FIG. 10 is a diagram showing an appearance example in a case in which the ultrasound image capturing apparatus 2A according to the present modification example is seen from a side surface. The ultrasound image capturing apparatus 2A shown in FIG. 10 is different from the ultrasound image capturing apparatus 2 shown in FIG. 4 in that a transducer 15B, a movement table 14B, and a transducer movement unit 9B are added, and other configurations are the same as the configurations of the ultrasound image capturing apparatus 2.

An output surface of the transducer 15B that outputs the ultrasonic waves is provided at a position facing the compression surface 47A of the compression plate 47, and is, for example, in contact with the compression surface 47A to capture the ultrasound image of the breast in contact with the compression surface 47A from the compression surface 47A side. An acoustic matching member such as gel or gel is applied to the output surface of the ultrasonic waves in the transducer 15B.

The transducer 15B is attached to the movement table 14B, and the transducer movement unit 9B moves the movement table 14B in the front-rear direction (in the example of FIG. 10, the direction represented by the arrow W1) under the control of the controller 20 via the transducer drive unit 22. Therefore, the transducer 15B attached to the movement table 14B is also moved in the front-rear direction in accordance with the movement of the movement table 14B. It should be noted that the transducer movement unit 9B may further move the transducer 15B in the chest wall surface direction while moving the transducer 15B in the front-rear direction. The transducer 15B is an example of a second transducer according to the present disclosure. The movement table 14B may have any shape as long as the movement table 14B is an object to which the transducer 15B is attached, like the movement table 14A.

The controller 20 controls each imaging start timing of the transducers 15A and 15B, and individually controls each movement range (that is, the imaging range of the breast) of the transducers 15A and 15B via the transducer drive unit 22. It should be noted that the ultrasound image capturing apparatus 2A need not comprise the movement table 14B and the transducer movement unit 9B that are examples of the movement mechanism of the transducer 15B. In a case in which the ultrasound image capturing apparatus 2A does not comprise the movement table 14B and the transducer movement unit 9B, the medical staff member may manually move the transducer 15B. In the present modification example, an example will be described in which the controller 20 individually controls each imaging start timing of the transducers 15A and 15B and the imaging range of the breast.

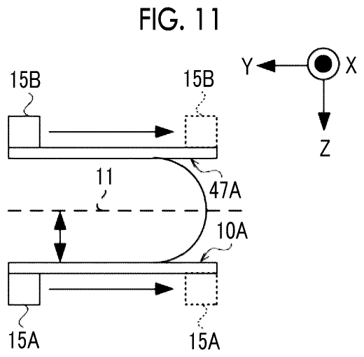

FIG. 11 is a diagram showing a control example of each imaging range of the transducers 15A and 15B, and is a diagram in which a configuration between the transducers 15A and 15B is extracted the ultrasound image capturing apparatus 2A in FIG. 10. For convenience of description, in FIG. 11, the transducer cover 16 is not shown.

As in the example shown in FIG. 11, the controller 20 performs control of the imaging range such that the transducers 15A and 15B image the same predetermined range (for example, a range including the entire breast) from different directions, specifically, from opposite directions. That is, in a case in which the compression surface 47A is seen from an upper position facing the compression surface 47A of the compression plate 47, the controller 20 performs movement control of the movement tables 14A and 14B such that the transducers 15A and 15B both move in the same range in which the breast is present.

In a case in which the breast is imaged by using the transducers 15A and 15B, the controller 20 sets a boundary surface 11 between the imaging surface 10A of the imaging table 10 and the compression surface 47A of the compression plate 47, for example, at a predetermined distance from the imaging surface 10A of the imaging table 10. Thereafter, the controller 20 performs control of combining the ultrasound image of the boundary surface 11, which is captured by the transducer 15A, from the imaging surface 10A of the imaging table 10 and the ultrasound image of the boundary surface 11, which is captured by the transducer 15B, from the compression surface 47A of the compression plate 47, to generate one ultrasound image for the same breast.

Hereinafter, the operation of the ultrasound image capturing apparatus 2A shown in FIG. 10 that captures the ultrasound image of the breast placed on the imaging surface 10A of the imaging table 10 by using the transducers 15A and 15B will be described.

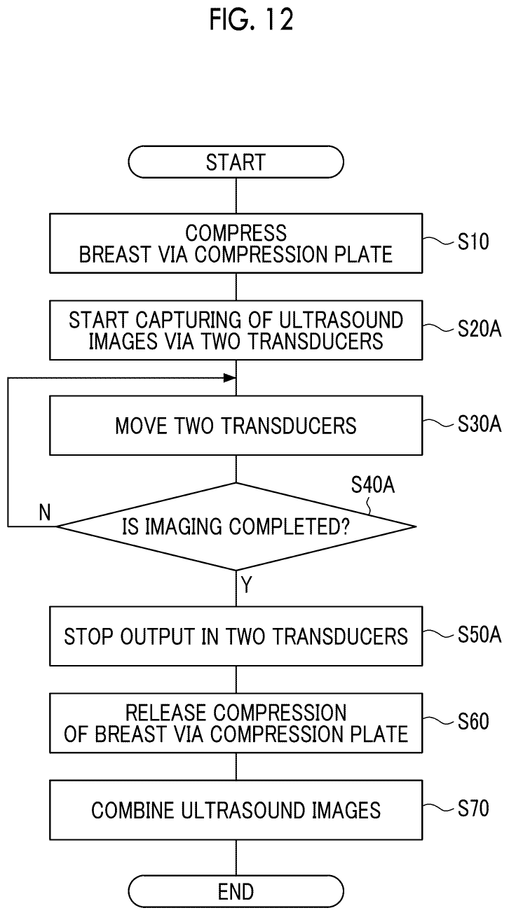

FIG. 12 is a flowchart showing an example of a flow of imaging processing executed by the ultrasound image capturing apparatus 2A in a case in which an instruction to start the capturing of the ultrasound image of the breast is received through an operation via the operation unit 27 performed by the medical staff member. The CPU 20A of the ultrasound image capturing apparatus 2A reads the control program 21 from the ROM 20B and executes the imaging processing. It is assumed that the subject has already placed the breast, which is the examination subject, on the imaging surface 10A of the imaging table 10.

The flowchart of the imaging processing shown in FIG. 12 is different from the flowchart of the imaging processing according to the first embodiment shown in FIG. 9 in that step S20, step S30, step S40, and step S50 are replaced with step S20A, step S30A, step S40A, and step S50A, respectively, and step S70 is newly added.

Therefore, hereinafter, the imaging processing in the present modification example will be described mainly with reference to the processing of steps S20A to S50A and step S70.

In a case in which the breast is compressed by the compression plate 47 in the processing of step S10, step S20A is executed.

In step S20A, the controller 20 performs control of the transducers 15A and 15B to output the ultrasonic waves from the transducers 15A and 15B, and starts the capturing of the ultrasound image of the breast.

In step S30A, the controller 20 performs control of the transducer movement units 9A and 9B via the transducer drive unit 22, and controls the movement ranges of the transducers 15A and 15B such that the transducers 15A and 15B capture the ultrasound images of the same predetermined range (for example, a range including the entire breast) of the breast.

It should be noted that the transducers 15A and 15B need not image the same location of the breast at the same time. For example, the controller 20 may capture the ultrasound image of the breast by using the transducer 15A, and then move the transducer 15B in the same range as the imaging range of the breast via the transducer 15A, to capture the ultrasound image of the breast by using the transducer 15B.

In step S40A, the controller 20 determines whether the transducers 15A and 15B have completed the capturing of the ultrasound image in the entire predetermined range. In a case in which the imaging is not yet completed, the processing proceeds to step S30A, and the processing of moving the transducers 15A and 15B to the designated position is repeatedly executed. As a result, the controller 20 can capture continuous ultrasound images of the breast in the same predetermined range by using each of the transducers 15A and 15B.

Meanwhile, in a case in which it is determined by the determination processing in step S40A that the capturing of the ultrasound image by the transducers 15A and 15B is completed, the processing proceeds to step S50A.

In step S50A, the controller 20 performs control of the transducers 15A and 15B to stop the output of the ultrasonic waves in the transducers 15A and 15B.

After the compression state of the breast is released by the processing of step S60, in step S70, the controller 20 combines the ultrasound image of the boundary surface 11, which is captured by the transducer 15A, from the imaging surface 10A of the imaging table 10 and the ultrasound image of the boundary surface 11, which is captured by the transducer 15B, from the compression surface 47A of the compression plate 47, to generate one ultrasound image for the same breast. It should be noted that the position of the boundary surface 11 need only be set by the medical staff member and stored in the storage unit 24 in advance.

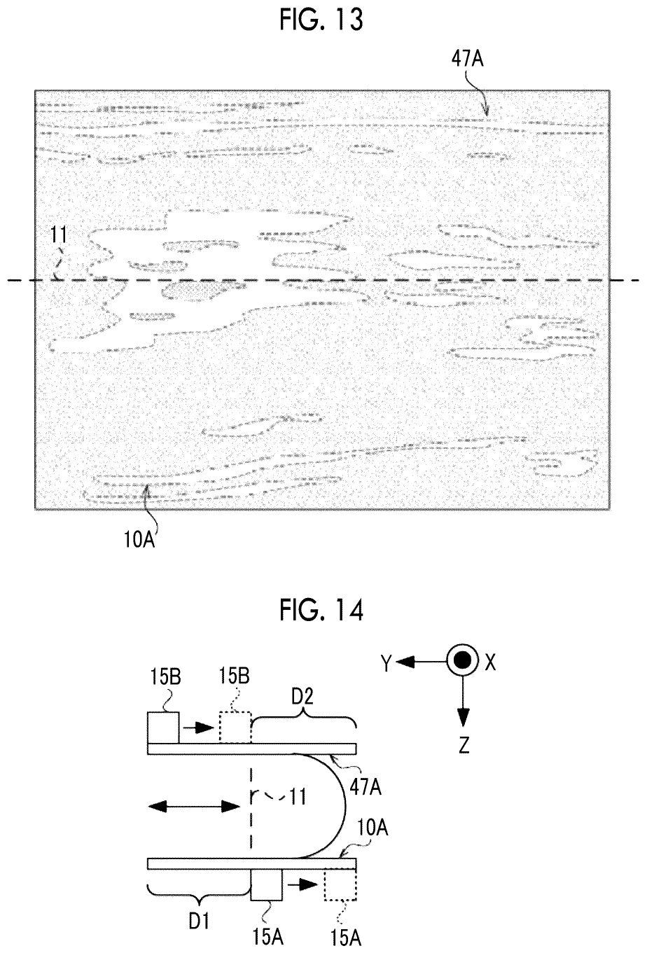

FIG. 13 is a diagram showing an example of the combined ultrasound image. For convenience of description, FIG. 13 shows the boundary surface 11, but it goes without saying that the boundary surface 11 is not shown in the actual ultrasound image. In this way, the imaging processing shown in FIG. 12 ends.

In the ultrasound image, the image quality tends to be lower for the object located farther from the output surface of the ultrasonic waves. Therefore, for example, in a case in which the ultrasound image of the breast is captured by using only the transducer 15A, the image quality is lower at a breast portion located at a place away from the imaging surface 10A in a vertical direction, that is, at a breast portion located at a place close to the compression surface 47A. On the contrary, for example, in a case in which the ultrasound image of the breast is captured by using only the transducer 15B, the image quality is degraded as a breast portion at a location away from the compression surface 47A in the vertical direction, that is, a breast portion at a location close to the imaging surface 10A.

However, in the ultrasound image capturing apparatus 2A according to the present modification example, the transducers 15A and 15B capture the ultrasound images of the breast from the imaging surface 10A of the imaging table 10 and the compression surface 47A of the compression plate 47, respectively. Although the ultrasound image of the region beyond the boundary surface 11 as seen from the transducer 15A has a lower image quality than the ultrasound image of the region up to the boundary surface 11, the ultrasound image of the region beyond the boundary surface 11 is replaced with the ultrasound image captured by the transducer 15B, and thus the image quality of the ultrasound image of the region beyond the boundary surface 11 is equivalent to the image quality of the ultrasound image of the region up to the boundary surface 11. Therefore, the ultrasound image having high accuracy and high contrast can be obtained as compared to a case in which the ultrasound image of the breast is captured by any one of the transducer 15A or the transducer 15B.

In this way, an imaging mode in which the transducers 15A and 15B image the same predetermined range from different directions and to combine the respective ultrasound images is referred to as a “high definition mode”.

In the above description, the transducers 15A and 15B image the predetermined same range of the breast, but the controller 20 of the ultrasound image capturing apparatus 2A can switch the imaging mode of the ultrasound image of the breast by controlling the imaging ranges of the transducers 15A and 15B.

FIG. 14 is a diagram showing a control example of each imaging range in the transducers 15A and 15B for implementing an imaging mode different from the high definition mode, and is a diagram in which a configuration between the transducers 15A and 15B is extracted from the ultrasound image capturing apparatus 2A in FIG. 10. For convenience of description, in FIG. 14, the transducer cover 16 is not shown.

As in the example shown in FIG. 14, the controller 20 performs control of the imaging range such that the transducers 15A and 15B image predetermined different ranges of the breast from different directions. That is, in a case in which the compression surface 47A is seen from an upper position facing the compression surface 47A of the compression plate 47, the controller 20 performs movement control of the movement tables 14A and 14B such that the movement ranges of the transducers 15A and 15B do not overlap each other.

In the example of FIG. 14, the transducer 15B images a range of a region D1 close to the chest wall, and the transducer 15A images a range of a region D2 close to the nipple, among ranges divided by the boundary surface 11 orthogonal to the imaging surface 10A and the compression surface 47A. It should be noted that the transducer 15A may image the range of the region D1 close to the chest wall, and the transducer 15B may image the range of the region D2 close to the nipple.

In the example of FIG. 14, in step S30A of the imaging processing shown in FIG. 12, the controller 20 performs control of the transducer movement units 9A and 9B via the transducer drive unit 22, and controls the movement ranges of the transducers 15A and 15B such that the transducers 15A and 15B capture the ultrasound images of predetermined different ranges of the breast assigned to the transducers 15A and 15B, respectively.

It should be noted that the transducers 15A and 15B need not capture the ultrasound images at the same time. For example, the controller 20 may capture the ultrasound image of the breast by using the transducer 15A and then capture the ultrasound image of the breast by using the transducer 15B.

In the determination processing of step S40A shown in FIG. 12, in a case in which it is determined that the transducers 15A and 15B have completed the capturing of the ultrasound image of the entire responsible range of the breast, the processing proceeds to step S70 after executing step S50A and step S60.

In step S70, the controller 20 combines the ultrasound image of the breast captured by using the transducer 15A and the ultrasound image of the breast captured by using the transducer 15B such that the division boundaries of the boundary surface 11 between the ultrasound images are adjacent to each other, to generate one ultrasound image of the same breast.

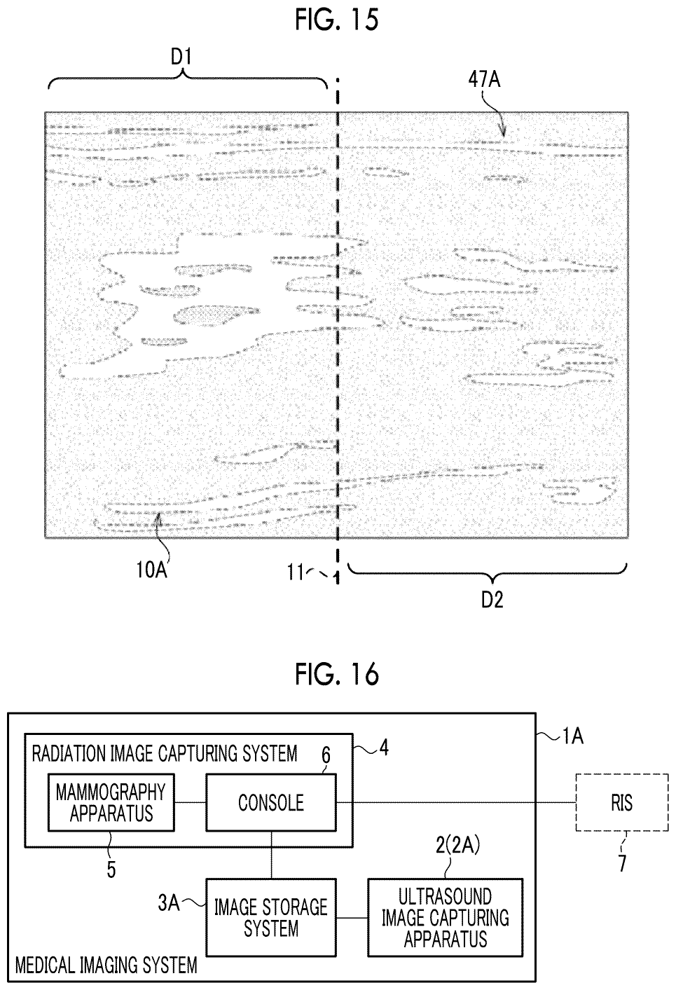

FIG. 15 is a diagram showing an example of the combined ultrasound image. The ultrasound image of the region D1 is, for example, the ultrasound image captured by the transducer 15B, and the ultrasound image of the region D2 is, for example, the ultrasound image captured by the transducer 15A. For convenience of description, FIG. 15 shows the boundary surface 11, but it goes without saying that the boundary surface 11 is not shown in the actual ultrasound image.

In a case in which the boundary surface 11 is set such that a shape and an area of the region D1 in contact with the compression surface 47A and a shape and an area of the region D2 in contact with the imaging surface 10A are the same, the movement ranges of the transducers 15A and 15B are the same. Therefore, in a case in which the capturing of the ultrasound images via the transducers 15A and 15B is started at the same time, the time required for capturing the ultrasound images of the region D1 and the region D2 is shortened to ½ of the time required for capturing the breast in the high definition mode.

For the above-described reason, the imaging mode shown in FIG. 11 is referred to as the high definition mode, whereas the imaging mode shown in FIG. 14 is referred to as a “high speed mode”.

In the high speed mode, it is possible to capture the ultrasound image of the same range of the breast in a time of ½ of the imaging time by using the high definition mode, but, in a case in which a specific point in the imaging range is focused, the ultrasound image is captured from the compression surface 47A to the imaging surface 10A at the point by any one of the transducer 15A or the transducer 15B. Therefore, since the ultrasound image in which a portion farther than in the high definition mode is imaged is included in the high speed mode, the ultrasound image captured in the high speed mode may have a lower image quality than the ultrasound image captured in the high definition mode.

### Second Embodiment

In the second embodiment, a medical imaging system 1A will be described in which a radiation image capturing system 4 is added to the medical imaging system 1 shown in FIG. 1.

FIG. 16 is a diagram showing a configuration example of the medical imaging system 1A according to the second embodiment. The medical imaging system 1A includes the ultrasound image capturing apparatus 2 or the ultrasound image capturing apparatus 2A, the radiation image capturing system 4, and an image storage system 3A obtained by expanding the image storage system 3 shown in FIG. 1. Further, the radiation image capturing system 4 includes the mammography apparatus 5 and the console 6.

The mammography apparatus 5 is an apparatus that irradiates the breast of the subject compressed by the compression plate 47 with radiation R (for example, X-rays: see FIG. 18), to capture a radiation image of the breast. The mammography apparatus 5 is implemented by a common housing with the ultrasound image capturing apparatus 2 shown in FIG. 4 or the ultrasound image capturing apparatus 2A shown in FIG. 10. That is, the mammography apparatus 5 also has the functions of the ultrasound image capturing apparatus 2 or the ultrasound image capturing apparatus 2A described in the first embodiment.

The console 6 is an operation console that is used to operate the mammography apparatus 5, and is connected to, for example, the mammography apparatus 5 and the image storage system 3A.

The image storage system 3A is a system that stores the ultrasound image captured by the ultrasound image capturing apparatus 2 or the ultrasound image capturing apparatus 2A and the radiation image captured by the mammography apparatus 5. The image storage system 3A extracts an ultrasound image and a radiation image corresponding to a request from the console 6 from among the stored ultrasound images and the stored radiation images, and transmits the extracted ultrasound image and the extracted radiation image to the console 6.

It should be noted that the console 6 receives an instruction from the medical staff member and notifies the controller 20 of the imaging order and various types of information acquired from a radiology information system (RIS) 7 via the communication line such as the LAN.

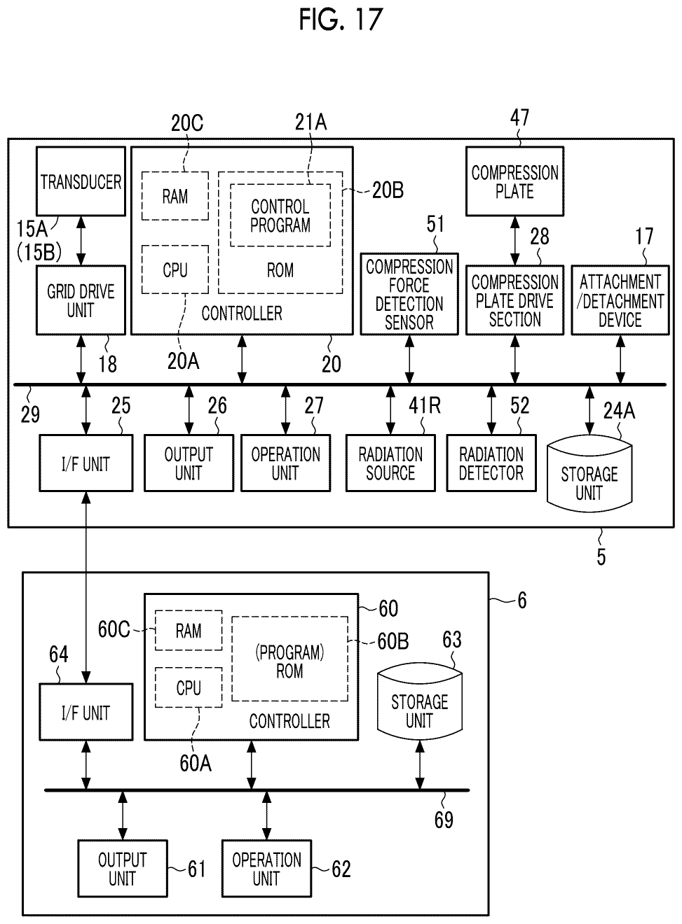

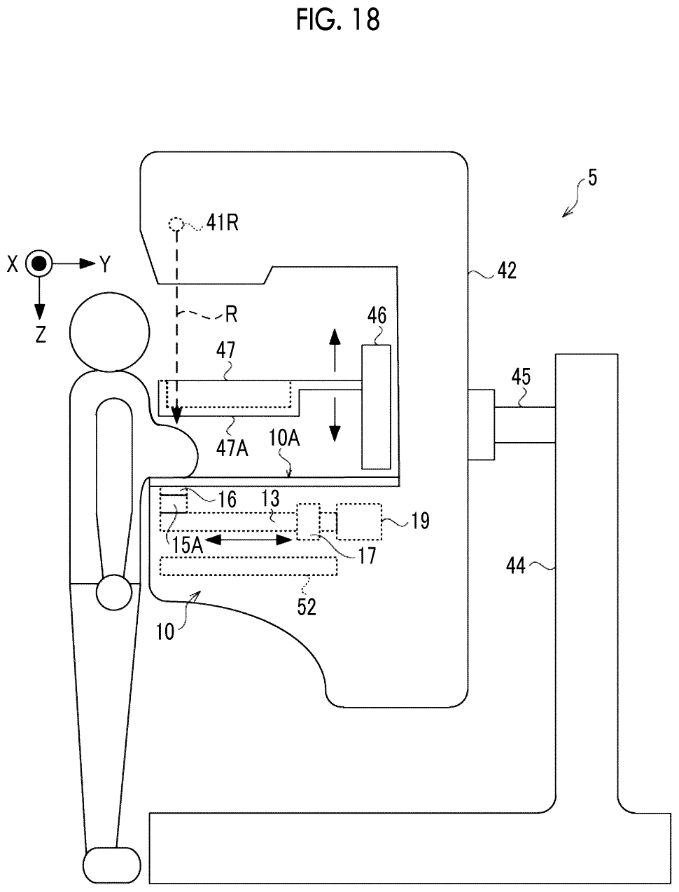

FIG. 17 is a diagram showing a functional configuration example of the mammography apparatus 5 and the console 6. Since the mammography apparatus 5 also has the functions of the ultrasound image capturing apparatus 2 or the ultrasound image capturing apparatus 2A, the mammography apparatus 5 comprises an attachment/detachment device 17, a radiation source 41R, and a radiation detector 52, in addition to the configurations of the ultrasound image capturing apparatuses 2 and 2A shown in FIG. 2. Further, the control program 21, the transducer drive unit 22, and the storage unit 24 in FIG. 2 are replaced with a control program 21A, the grid drive unit 18, and a storage unit 24A, respectively.

The radiation source 41R irradiates the breast with the radiation R under the control of the controller 20 that receives an instruction from the console 6.

The radiation detector 52 detects the radiation R that has passed through the breast which is the examination subject.

The storage unit 24A stores the captured ultrasound images and the captured radiation images, along with various information.

The attachment/detachment device 17 is a device that attaches and detaches the transducer 15A to and from a grid 13 (see FIG. 18). Details of the grid 13 and the attachment/detachment device 17 will be described later.

The grid drive unit 18 controls a grid movement unit 19 (see FIG. 18) disposed inside the imaging table 10 in response to an instruction from the controller 20 to move the grid 13 in at least one of the chest wall surface direction or the front-rear direction.

It should be noted that the compression plate 47 in the mammography apparatus 5 is made of a material having excellent transmittance for the radiation R. Further, it is preferable that the compression plate 47 is made of a material through which ultrasonic waves are easily propagated. Examples of the material forming the compression plate 47 include a resin such as polymethylpentene, polycarbonate, acrylic, and polyethylene terephthalate. In particular, polymethylpentene is suitable as the material forming the compression plate 47 since polymethylpentene has low rigidity, high elasticity, and high flexibility and has suitable values for acoustic impedance that affects the reflectance of the ultrasonic waves and an attenuation coefficient that affects the attenuation of ultrasonic waves. It should be noted that the member forming the compression plate 47 is not limited to the above-described example. For example, the member forming the compression plate 47 may be a film-shaped member.

The control program 21A is a program that is read by the CPU 20A to perform control related to the capturing of the ultrasound image and control related to the capturing of the radiation image.

Meanwhile, the console 6 is configured by, for example, a server computer. As shown in FIG. 17, the console 6 comprises a controller 60, an output unit 61, an operation unit 62, a storage unit 63, and an I/F unit 64. The controller 60, the output unit 61, the operation unit 62, the storage unit 63, and the I/F unit 64 are connected to each other via a bus 69 such that various types of information can be exchanged.

The controller 60 controls the overall operation of the console 6. The controller 60 comprises a CPU 60A, a ROM 60B, and a RAM 60C. Various programs and the like executed by the CPU 60A are stored in the ROM 60B in advance. The RAM 60C is used as a transitory work area of the CPU 60A.

The output unit 61 outputs information processed by the controller 60 to the medical staff member.

The operation unit 62 is used by the medical staff member to input instructions, various types of information, and the like related to the capturing of the radiation image, including an instruction to perform the irradiation with the radiation R. Therefore, the operation unit 62 includes at least an irradiation instruction button that is pressed by the medical staff member to input the instruction to perform the irradiation with the radiation R. There is no restriction on an operation form in the operation unit 62, and, for example, the operation can be received by a switch, a touch panel, a touch pen, a keyboard, a mouse, or the like.

The storage unit 63 stores the radiation images captured by the mammography apparatus 5, various types of information, and the like. The storage unit 63 is an example of a storage device that holds the stored information even in a case in which power supplied to the storage unit 63 is cut off, and, for example, a semiconductor memory, such as an SSD, is used, or a hard disk may be used.

The I/F unit 64 transmits and receives various types of information to and from the mammography apparatus 5 connected to, for example, the communication line such as the LAN, the RIS 7, and the image storage system 3A via wireless communication or wired communication. For example, the console 6 receives the radiation image captured by the mammography apparatus 5 via the I/F unit 64, transmits the received radiation image to the image storage system 3A via the I/F unit 64, and stores the radiation image in the image storage system 3A.

FIG. 18 is a diagram showing an appearance example in a case in which the mammography apparatus 5 is seen from a side surface. In the mammography apparatus 5, the ultrasound image and the radiation image of the breast are also captured in a state in which the subject places the breast on the imaging surface 10A of the imaging table 10. The mammography apparatus 5 can capture the image of the breast of the subject in a state in which the subject is sitting on a chair (including a wheelchair) or the like (sitting state) in addition to a state in which the subject is standing (standing state).

In the following description, the apparatus configuration of the mammography apparatus 5 based on the ultrasound image capturing apparatus 2 shown in FIG. 4 will be described, but the mammography apparatus 5 may be configured based on the ultrasound image capturing apparatus 2A shown in FIG. 10.

In the mammography apparatus 5 shown in FIG. 18, the radiation source 41R, the radiation detector 52, and the attachment/detachment device 17 are added to the apparatus configuration of the ultrasound image capturing apparatus 2 shown in FIG. 4. In addition, the grid 13 is used as the movement table 14A of the ultrasound image capturing apparatus 2 shown in FIG. 4.

The radiation source 41R is provided at a position of the arm part 42 facing the imaging surface 10A of the imaging table 10.

The radiation detector 52 is disposed inside the imaging table 10 on a rear side with respect to the transducer 15A in a case in which the radiation source 41R is seen in a direction of the imaging surface 10A, and detects the radiation R that has passed through the breast.

In addition, the grid 13 is disposed in a space inside the imaging table 10. In a case in which the radiation R is transmitted through the breast, scattered rays may be generated, but, in a case in which the scattered rays are incident on the radiation detector 52, a portion that should not be exposed to light is exposed to light, and the image quality of the radiation image may be degraded. The grid 13 functions as a filter that reduces the scattered rays incident on the radiation detector 52 as compared to before the grid 13 is installed, before the scattered rays are incident on the radiation detector 52. Therefore, for example, the grid 13 is disposed to cover the radiation detector 52 between the imaging surface 10A on which the breast is placed and the radiation detector 52.

However, in a case in which the radiation image is captured by the tomosynthesis, the radiation R that is originally supposed to pass through the grid 13 other than the scattered rays may be blocked by the grid 13, and thus it is necessary to retract the grid 13 to the outside of the radiation detection range of the radiation detector 52. Therefore, the mammography apparatus 5 comprises the grid movement unit 19 inside the imaging table 10 and includes a movement mechanism that moves the grid 13 in at least one of the chest wall surface direction or the front-rear direction.

As described above, for example, in a case in which the ultrasound image of the breast is captured by the ultrasound image capturing apparatus 2, the transducer 15A is moved by using the movement table 14A, but the grid 13 is not required because the radiation R is not emitted during the capturing of the ultrasound image. Therefore, in a case in which the transducer 15A is attached to the grid 13, the grid 13 can be used as the movement table 14A that moves the position of the transducer 15A, and it is not necessary to separately provide the transducer movement unit 9A in the mammography apparatus 5. That is, in the mammography apparatus 5, the grid 13 and the grid movement unit 19 are used as a movement mechanism that moves the transducer 15A in at least one of the chest wall surface direction or the front-rear direction.

In this case, in a case in which the transducer 15A is attached in a state of being fixed to the grid 13, in a case in which the radiation image of the breast is captured, the transducer 15A is reflected in the radiation image. Therefore, the mammography apparatus 5 comprises the attachment/detachment device 17 that detaches and attaches the transducer 15A to and from the grid 13.

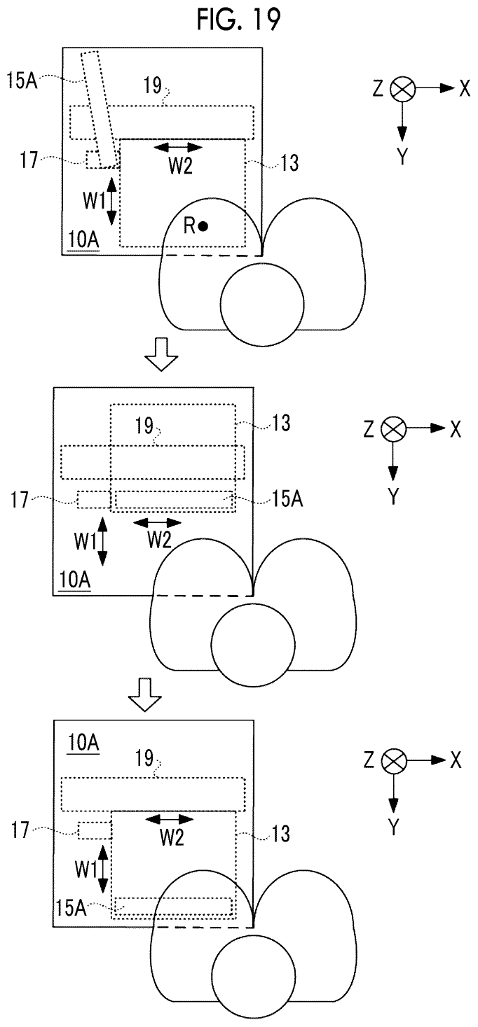

FIG. 19 is a diagram showing an operation example of the attachment/detachment device 17. As shown in FIG. 19, in the mammography apparatus 5, the transducer 15A is not attached to the grid 13 during the capturing of the radiation image.

In a case in which the capturing of the radiation image ends, the grid movement unit 19 moves the grid 13 in, for example, the front-rear direction (in the example of FIG. 19, the direction represented by the arrow W1) to move the grid 13 away from the subject.

In a case in which the grid 13 is moved to a predetermined position (attachment/detachment position) as the attachment/detachment location of the transducer 15A, the attachment/detachment device 17 attaches the transducer 15A to the grid 13 such that the longitudinal direction of the transducer 15A is along the chest wall surface direction (in the example of FIG. 19, a direction represented by the arrow W2).

Then, the grid movement unit 19 moves the grid 13 in the front-rear direction such that the transducer 15A approaches the subject, and captures the ultrasound image of the breast while performing the scanning of the transducer 15A.

In a case in which the capturing of the ultrasound image ends, the grid movement unit 19 moves the grid 13 in, for example, the front-rear direction to move the grid 13 away from the subject. In a case in which the grid 13 returns to the attachment/detachment position of the transducer 15A, the attachment/detachment device 17 detaches the transducer 15A from the grid 13.

It should be noted that there is no restriction on the method of attaching and detaching the transducer 15A in the attachment/detachment device 17, and a known attachment and detachment method can be used.

Hereinafter, the operation of the mammography apparatus 5 that captures the ultrasound image and the radiation image of the breast will be described in detail.

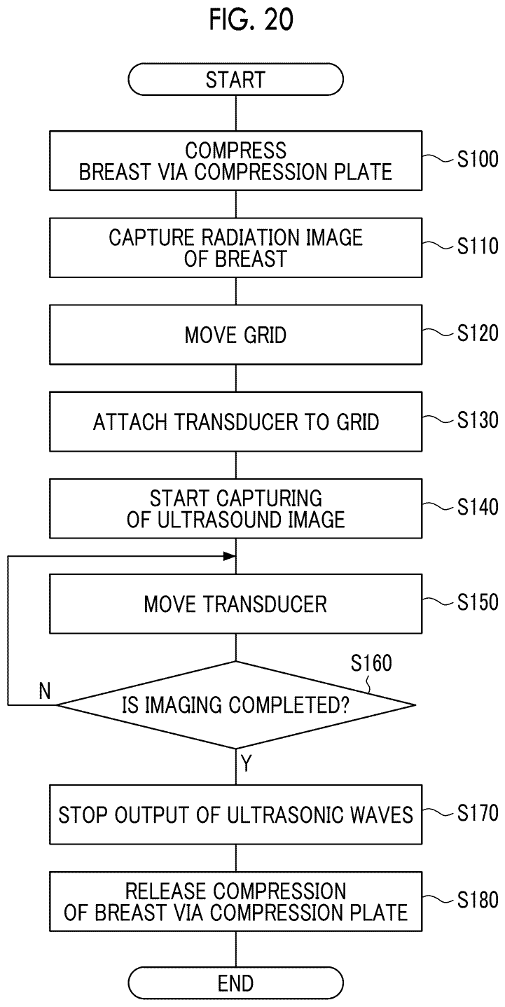

FIG. 20 is a flowchart showing an example of a flow of the imaging processing executed by the mammography apparatus 5 in a case in which an instruction to start the capturing of the ultrasound image and the radiation image for the breast is received by the console 6 through the operation of the medical staff member. The CPU 20A of the mammography apparatus 5 reads the control program 21A from the ROM 20B and executes the imaging processing. As shown in FIG. 18, the subject is in a state in which the breast as the examination subject is placed on the imaging surface 10A of the imaging table 10. Further, the transducer 15A is in a state of being not attached to the grid 13.

First, in step S100, the controller 20 performs control of the compression plate drive unit 46 via the compression plate drive section 28, and moves the compression plate 47 in the compression direction to compress the breast with the compression plate 47 and press the breast against the imaging surface 10A.

In step S110, the controller 20 controls the grid movement unit 19 via the grid drive unit 18 to move the grid 13 in the front-rear direction such that the grid 13 covers the radiation detector 52. Thereafter, the controller 20 controls the radiation source 41R to start the irradiation with the radiation R, and captures the radiation image of the breast compressed by the compression plate 47. In this case, since slits formed by a lead wire and an intermediate substance forming the grid 13 may be reflected in the radiation image, it is preferable that the controller 20 controls the grid movement unit 19 to capture the radiation image of the breast while swinging the grid 13 in the chest wall surface direction.

After the capturing of the radiation image of the breast ends, in step S120, the controller 20 controls the grid movement unit 19 via the grid drive unit 18 to move the grid 13 until one end of the grid 13 closest to the subject comes to the attachment/detachment position.

In step S130, the controller 20 controls the attachment/detachment device 17 to attach the transducer 15A that has been retracted to the grid 13.

In step S140, the controller 20 performs control of the transducer 15A to output the ultrasonic waves from the transducer 15A and starts the capturing of the ultrasound image of the breast.

In step S150, the controller 20 performs control of the grid movement unit 19 via the grid drive unit 18 to move the transducer 15A along the front-rear direction. Therefore, the controller 20 can capture continuous ultrasound images of the entire breast.

In step S160, the controller 20 determines whether the capturing of the ultrasound image in the planned range is completed. In a case in which the imaging is not yet completed, the processing proceeds to step S150, and the processing of moving the transducer 15A to the designated position is repeatedly executed.

On the other hand, in a case in which it is determined by the determination processing of step S160 that the capturing of ultrasound images is completed, the processing proceeds to step S170.

In step S170, the controller 20 performs control of the transducer 15A to stop the output of the ultrasonic waves.

In step S180, the controller 20 moves the compression plate 47 in the compression release direction and releases the compression of the breast via the compression plate 47 by controlling the compression plate drive unit 46 via the compression plate drive section 28. In this way, the imaging processing shown in FIG. 20 ends.

In this way, the mammography apparatus 5 uses the grid 13 as the movement table 14A of the transducer 15A. In a case of capturing the radiation image of the breast, the mammography apparatus 5 attaches the transducer 15A from the grid 13 by using the attachment/detachment device 17 and retracts the transducer 15A to a position outside the radiation detection range of the radiation detector 52. In addition, in a case of capturing the ultrasound image of the breast, the mammography apparatus 5 attaches the transducer 15A, which has been retracted by the attachment/detachment device 17, to the grid 13. Therefore, even in a case in which the transducer 15A is built in the imaging table 10 to capture the ultrasound image of the breast from the imaging surface 10A, the transducer 15A can be prevented from being reflected in the radiation image.

It should be noted that the attachment direction of the transducer 15A to the grid 13 and the movement direction of the grid 13 are examples, and, as shown in FIG. 6, the transducer 15A may be attached to the grid 13 instead of the movement table 14A such that the longitudinal direction of the transducer 15A is along the front-rear direction (in the example of FIG. 6, the direction represented by the arrow W1), and the grid 13 may be moved in the chest wall surface direction (in the example of FIG. 6, the direction represented by the arrow W2).

As described above, the forms of the medical imaging systems 1 and 1A have been described with the embodiments, but the disclosed forms of the medical imaging systems 1 and 1A are examples, and the forms of the medical imaging systems 1 and 1A are not limited to the range described in the embodiments. Various modifications and improvements can be added to the embodiments without departing from the gist of the present disclosure, and the embodiments to which the modifications or improvements are added are also included in the technical scope of the present disclosure.

For example, the processing order in the flowchart of each imaging processing shown in FIGS. 9, 12, and 20 may be changed without departing from the gist of the present disclosure.

In each of the above-described embodiments, as an example, a form has been described in which each imaging processing is implemented by software processing. On the other hand, processing equivalent to the flowchart of the imaging processing may be implemented by hardware. In this case, the processing speed can be increased as compared to a case in which the imaging processing is implemented by the software processing.

In the embodiments above, the processor is a processor in a broad sense, and includes a general-purpose processor (for example, the CPU 20A) or a dedicated processor (for example, a graphics processing unit (GPU), an application-specific integrated circuit (ASIC), a field programmable gate array (FPGA), a programmable logic device, and the like).

In addition, the operation of the processor in each of the above-described embodiments may be performed not only by one processor but also by cooperation of a plurality of processors provided at physically separated positions. In addition, the order of the operations of the processor is not limited to only the order described in each of the above-described embodiments, and may be changed as appropriate.

In each of the above-described embodiments, examples have been described in which the control programs 21 and 21A are stored in the ROM 20B. However, the storage destination of the control programs 21 and 21A is not limited to the ROM 20B. The control programs 21 and 21A can also be provided in a form of being recorded in a computer-readable storage medium.

For example, the control programs 21 and 21A may be provided in a form of being recorded on an optical disk, such as a compact disk read-only memory (CD-ROM), a digital versatile disk read-only memory (DVD-ROM), and a blue ray disk. In addition, the control programs 21 and 21A may be provided in a form of being recorded in a portable semiconductor memory, such as a universal serial bus (USB) memory and a memory card. The ROM 20B, the CD-ROM, the DVD-ROM, the blue ray disk, the USB, and the memory card are examples of a non-transitory storage medium.

Further, the controller 20 may download the control programs 21 and 21A from an external apparatus connected to the communication line via the I/F unit 25, to store the downloaded control programs 21 and 21A in the ROM 20B of the controller 20.

In regard with the above-described embodiments, the following supplementary notes are further disclosed.

### Supplementary Note 1

An ultrasound image capturing apparatus comprising: an imaging table in which a first transducer that transmits an ultrasonic wave toward a contact surface in contact with a breast, to capture an ultrasound image of the breast, and a movement mechanism that moves the first transducer in a direction along the contact surface with the breast are built in advance, in which the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets.

### Supplementary Note 2

The ultrasound image capturing apparatus according to supplementary note 1, in which the first transducer has a shape that extends longer in a chest wall surface direction along a chest wall of a subject than in a front-rear direction along a direction intersecting the chest wall of the subject, and the movement mechanism moves the first transducer in the front-rear direction.

### Supplementary Note 3

The ultrasound image capturing apparatus according to supplementary note 2, in which the movement mechanism further moves the first transducer in the chest wall surface direction.

### Supplementary Note 4

The ultrasound image capturing apparatus according to supplementary note 1, in which the first transducer has a shape that extends longer in a front-rear direction along a direction intersecting a chest wall of a subject than in a chest wall surface direction along the chest wall of the subject, and the movement mechanism moves the first transducer in the chest wall surface direction.

### Supplementary Note 5

The ultrasound image capturing apparatus according to supplementary note 4, in which the movement mechanism further moves the first transducer in the front-rear direction.

### Supplementary Note 6

The ultrasound image capturing apparatus according to any one of supplementary notes 1 to 5, in which, in the contact surface of the imaging table, the carbon fiber sheets are laminated such that the acoustic impedance is decreased from a carbon fiber sheet in contact with the first transducer toward a carbon fiber sheet in contact with the breast.

### Supplementary Note 7

The ultrasound image capturing apparatus according to supplementary note 6, in which a fiber direction of at least one carbon fiber sheet among the plurality of laminated carbon fiber sheets is disposed along a chest wall surface direction along a chest wall of a subject.

### Supplementary Note 8

The ultrasound image capturing apparatus according to supplementary note 7, in which the fiber direction of each of the carbon fiber sheet in contact with the first transducer and the carbon fiber sheet in contact with the breast is disposed along the chest wall surface direction.

### Supplementary Note 9

The ultrasound image capturing apparatus according to any one of supplementary notes 1 to 8, further comprising: a second transducer that captures an ultrasound image of the breast from a compression surface of a compression plate that compresses the breast to bring the breast into close contact with the contact surface of the imaging table; and a controller that captures the ultrasound images of the breast by using the first transducer and the second transducer.

### Supplementary Note 10

The ultrasound image capturing apparatus according to supplementary note 9, in which the controller switches an imaging mode of the ultrasound image of the breast by controlling imaging ranges of the first transducer and the second transducer.

### Supplementary Note 11

The ultrasound image capturing apparatus according to supplementary note 10, in which the controller sets a boundary surface between the contact surface of the imaging table and the compression surface of the compression plate at a predetermined distance from the contact surface of the imaging table, and then performs control of the imaging ranges such that the first transducer and the second transducer image the same range of the breast and performs control of combining the ultrasound image, which is captured by the first transducer, from the contact surface of the imaging table to the boundary surface and the ultrasound image, which is captured by the second transducer, from the compression surface of the compression plate to the boundary surface, to generate one ultrasound image of the same breast.

### Supplementary Note 12

The ultrasound image capturing apparatus according to supplementary note 10, in which the controller performs control of the imaging ranges such that the first transducer and the second transducer image different ranges.

### Supplementary Note 13

A mammography apparatus comprising: the ultrasound image capturing apparatus according to any one of supplementary notes 1 to 12.

### Supplementary Note 14

The mammography apparatus according to supplementary note 13, in which a grid that is provided between the contact surface of the imaging table and a radiation detector and that reduces an amount of scattered rays, which are generated by scattering of radiation emitted from a radiation source via the breast, incident on the radiation detector as compared to before installation is used as the movement mechanism.

### Supplementary Note 15

The mammography apparatus according to supplementary note 14, in which the imaging table includes an attachment/detachment device that attaches the first transducer to the grid in a case in which the grid is moved to a predetermined position and that detaches the first transducer from the grid in a case in which the grid to which the first transducer is attached returns to the position.

### Supplementary Note 16

A control program for causing a computer to execute a process, with respect to an ultrasound image capturing apparatus including an imaging table in which a first transducer that transmits an ultrasonic wave toward a contact surface in contact with a breast, to capture an ultrasound image of the breast, and a movement mechanism that moves the first transducer are built in advance, in which the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets, the process comprising: controlling the movement mechanism to move the first transducer in a direction along the contact surface of the imaging table with the breast and controlling the first transducer to capture the ultrasound image of the breast from the contact surface of the imaging table with the breast.

What is claimed is:

1. An ultrasound image capturing apparatus comprising:
an imaging table in which
a first transducer that transmits an ultrasonic wave toward a contact surface in contact with a breast, to capture an ultrasound image of the breast, and
a movement mechanism that moves the first transducer in a direction along the contact surface with the breast
are built in advance,
wherein the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets.

2. The ultrasound image capturing apparatus according to claim 1,
wherein the first transducer has a shape that extends longer in a chest wall surface direction along a chest wall of a subject than in a front-rear direction along a direction intersecting the chest wall of the subject, and
the movement mechanism moves the first transducer in the front-rear direction.

3. The ultrasound image capturing apparatus according to claim 2,
wherein the movement mechanism further moves the first transducer in the chest wall surface direction.

4. The ultrasound image capturing apparatus according to claim 1,
wherein the first transducer has a shape that extends longer in a front-rear direction along a direction intersecting a chest wall of a subject than in a chest wall surface direction along the chest wall of the subject, and
the movement mechanism moves the first transducer in the chest wall surface direction.

5. The ultrasound image capturing apparatus according to claim 4,
wherein the movement mechanism further moves the first transducer in the front-rear direction.

6. The ultrasound image capturing apparatus according to claim 1,
wherein, in the contact surface of the imaging table, the carbon fiber sheets are laminated such that the acoustic impedance is decreased from a carbon fiber sheet in contact with the first transducer toward a carbon fiber sheet in contact with the breast.

7. The ultrasound image capturing apparatus according to claim 6,
wherein a fiber direction of at least one carbon fiber sheet among the plurality of laminated carbon fiber sheets is disposed along a chest wall surface direction along a chest wall of a subject.

8. The ultrasound image capturing apparatus according to claim 7,
wherein the fiber direction of each of the carbon fiber sheet in contact with the first transducer and the carbon fiber sheet in contact with the breast is disposed along the chest wall surface direction.

9. The ultrasound image capturing apparatus according to claim 1, further comprising:
a second transducer that captures an ultrasound image of the breast from a compression surface of a compression plate that compresses the breast to bring the breast into close contact with the contact surface of the imaging table; and
a controller that captures the ultrasound images of the breast by using the first transducer and the second transducer.

10. The ultrasound image capturing apparatus according to claim 9,
wherein the controller switches an imaging mode of the ultrasound image of the breast by controlling imaging ranges of the first transducer and the second transducer.

11. The ultrasound image capturing apparatus according to claim 10,
wherein the controller sets a boundary surface between the contact surface of the imaging table and the compression surface of the compression plate at a predetermined distance from the contact surface of the imaging table, and then performs control of the imaging ranges such that the first transducer and the second transducer image the same range of the breast and performs control of combining the ultrasound image, which is captured by the first transducer, from the contact surface of the imaging table to the boundary surface and the ultrasound image, which is captured by the second transducer, from the compression surface of the compression plate to the boundary surface, to generate one ultrasound image of the same breast.

12. The ultrasound image capturing apparatus according to claim 10,
wherein the controller performs control of the imaging ranges such that the first transducer and the second transducer image different ranges.

13. A mammography apparatus comprising:
the ultrasound image capturing apparatus according to claim 1.

14. The mammography apparatus according to claim 13,
wherein a grid that is provided between the contact surface of the imaging table and a radiation detector and that reduces an amount of scattered rays, which are generated by scattering of radiation emitted from a radiation source via the breast, incident on the radiation detector as compared to before installation is used as the movement mechanism.

15. The mammography apparatus according to claim 14,
wherein the imaging table includes an attachment/detachment device that attaches the first transducer to the grid in a case in which the grid is moved to a predetermined position and that detaches the first transducer from the grid in a case in which the grid to which the first transducer is attached returns to the position.

16. A non-transitory computer-readable storage medium storing a control program executable by computer to execute a process, with respect to an ultrasound image capturing apparatus including an imaging table in which a first transducer that transmits an ultrasonic wave toward a contact surface in contact with a breast, to capture an ultrasound image of the breast, and a movement mechanism that moves the first transducer are built in advance, in which the contact surface of the imaging table is composed of a plurality of laminated carbon fiber sheets having different acoustic impedances, and at least one of the plurality of carbon fiber sheets has higher rigidity with respect to the contact with the breast than the other laminated carbon fiber sheets, the process comprising:
controlling the movement mechanism to move the first transducer in a direction along the contact surface of the imaging table with the breast and controlling the first transducer to capture the ultrasound image of the breast from the contact surface of the imaging table with the breast.
